(19) United States
(12) Patent Application Publication
(10) Pub. No.: US 2009/0011514 A1
(43) Pub. Date: Jan. 8, 2009

(54) Dna Crosslinking for Primer Extension Assays

(76) Inventors: Peter Williams, Phoenix, AZ (US); Sergei Aksyonov, Tempe, AZ (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 11/884,908

(22) PCT Filed: Feb. 22, 2006
(86) PCT No.: PCT/US2006/006183

§ 371 Date: Aug. 11, 2008

Related U.S. Application Data
(60) Provisional application No. 60/655,000, filed on Feb. 22, 2005.

(51) Int. Cl. G01N 33/00

(52) U.S. Cl. 436/94

(57) ABSTRACT

Provided herein are compositions and methods for inhibiting false signals associated with mispriming in primer extension assays.

### CROSS-REFERENCE TO RELATED APPLICATIONS

This application claims benefit of U.S. Provisional Application No. 60/655,000, filed Feb. 22, 2005, which is hereby incorporated herein by reference in its entirety.

### STATEMENT REGARDING FEDERALLY SPONSORED RESEARCH

This invention was made with government support under Grant R01 HG003567 awarded by NIH. The government has certain rights in the invention.

### BACKGROUND OF THE INVENTION

Sequencing-by-extension (SBE) involves supplying a primer and a template in the presence of polymerase enzyme, with only one type of nucleotide at a time, and detecting a signal that indicates whether or not a reaction has occurred: if a positive signal is detected it means that the base on the nucleotide supplied was complementary to the next template base, thus identifying that template base.

For polymerase-catalyzed extension to occur it is essential only that a small number of successive nucleotides of the primer, including the 3′ terminal nucleotide, are all complementary to, and are hybridized to some region of, the template. The minimum length of this complementary region varies depending upon the polymerase type and conditions (salt concentration, temperature etc) but can be as low as 4 nucleotides. It is quite possible for there to exist accidental complementary overlaps of 4 nucleotides between the 3′ end of the primer (or of the template) and some region on the template that is not the target region for sequencing. Polymerase-catalyzed extension from any such accidental hybridization is called “mispriming”. Note that each successive extension of the misprimed region makes a longer complementary length that has a higher probability of competing with the correct primer/template matched region.

Despite the expected high discrimination power of primer extension, in practice a false signal is often observed (Gemignani et al, 2002; Aksyonov et al, in prep). It is supposed that one of the sources of such false signal is mispriming caused by undesirable secondary structures formed by DNA (Nikiforov et al, 1994; Mitra et al, 2003). If DNA polymerase can use any 3′-end perfectly hybridized to a complementary strand then not only primer can be extended, but the template can be, too, as shown in FIG. 1. Additionally, both primer and template can be aligned with each other or with themselves or with a neighboring immobilized primer molecule or a template hybridized to the latter, through hairpins and bulges and all these secondary structures potentially can be used by DNA polymerase, so long as a short complementary region exists including the 3′ end of one of the strands. This causes erroneous signals (false positives). Mispriming also weakens or eliminates the correct signal, causing false negatives. A partial cure is found in careful design of the primer sequence and careful choice of the targeted template region. Modern software and availability of genome databases allow such primer design, which can minimize but not eliminate mispriming. But such an approach strongly restricts the applicability of primer extension. Only certain template sites can be examined and many others cannot. In addition, such an approach cannot eliminate mispriming resulting from accidental self-hybridization of the overhanging single-stranded 3′ terminus of the template strand, through formation of a hairpin structure. Clearly, a more universal method for mispriming suppression would be very welcome in the practice of primer extension.

### BRIEF SUMMARY OF THE INVENTION

In accordance with the purpose of this invention, as embodied and broadly described herein, this invention relates to compositions and methods for inhibiting mispriming associated with primer extension assays.

The invention also relates to compositions and methods that allow retention of the primer-template as a duplex for an extended time through many reaction and washing cycles, in order to increase the number of primer extension steps and, therefore, to increase the SBE read length. The provided compositions and methods inhibit detachment and loss of the template. This feature is applicable to any sequencing method that involves repeated manipulation of the same DNA molecule (such as single molecule sequencing-by-extension).

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows DNA strand crosslinking on a microarray and the ability to retain the hybridized template as the temperature is raised to melt or dissociate a hairpin structure formed at the 3′ terminus of the overhanging single-stranded template.

FIG. 2 is a diagram of the use of psoralin-mediated crosslinking in DNA sequencing-by-synthesis.

FIG. 3 shows observed fluorescence images from psoralin-mediated crosslinking in DNA sequencing-by-synthesis.

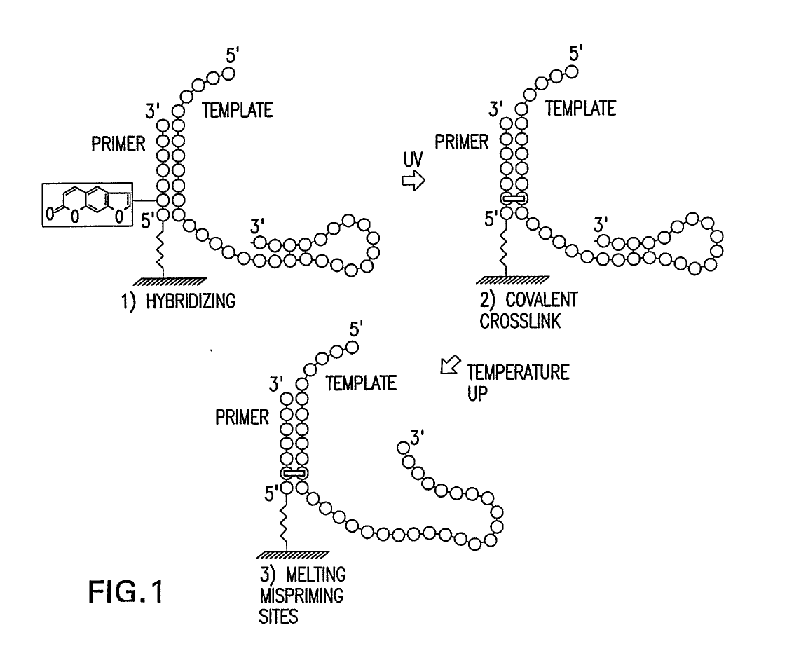

### DETAILED DESCRIPTION OF THE INVENTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, specific sequences of oligonucleotides, or to particular reagents unless otherwise specified, and, as such, may vary. It is also to be understood that the terminology used herein is for the purpose of describing particular embodiments only and is not intended to be limiting.

### A. METHOD

Provided herein are compositions and methods for inhibiting or reducing mispriming associated with primer extension assays. Primer extension refers to the incorporation of nucleotides such as deoxyribonucleotides into a primer-template duplex, wherein the nucleotide attaches to the 3′ end of the primer strand and is complementary to the nucleotide on the opposing template strand. Mispriming refers to the formation of DNA duplexes other than those that result from the desired primer and template being fully aligned and perfectly complementary. For example, secondary structures can form within the template strand. This mispriming can be prevented or reduced by, for example, increasing the stringency of hybridization during the primer extension reaction, for example by raising the temperature or reducing the salt concentration of the reaction mixture to destabilize short regions where either the primer and template strands are accidentally complementary or primer and/or template are accidentally self-complementary. However, increased stringency would normally also reduce the stability of the desired, fully complementary primer-template duplex and thereby lead to the dissociation of template from the primer, stopping the SBE process. Thus, provided herein is a method of stabilizing the primer-template duplex, which can be combined with increased hybridization stringency conditions to prevent or reduce mispriming.

1. Crosslinking

As disclosed herein, the primer-template duplex can be stabilized by covalently crosslinking the primer and template DNA strands together. Thus, provided herein is a method of preventing or reducing mispriming comprising crosslinking the primer to the template thereby allowing the primer extension reaction to be carried out under conditions of increased hybridization stringency. Such crosslinking can be accomplished in any suitable way and by any suitable means. For example, a crosslinking moiety can be used to crosslink the primer and template. For this purpose, the primer can comprise a crosslinking moiety. The crosslinking moiety can be directly incorporated into the primer at the time of synthesis through the use of appropriately modified nucleoside or nucleotide derivatives. Alternatively, the crosslinking molecule can be introduced into the primer-template duplex after hybridization, for example using soluble derivatives of the crosslinking molecule followed by photochemical or chemical activation. In some cases, the crosslinking moiety can be incorporated into a primer or template enzymatically by ligating an appropriately modified oligonucleotide which contains a crosslinking moiety.

Thus, crosslinking of the primer-template duplex can also involve the use of an oligonucleotide comprising a crosslinking moiety that is ligated to the primer or the template. For example, a universal primer can be ligated to a primer that is complementary to the template. In one aspect, the universal primer comprises the crosslinking moiety. In another aspect, the complementary primer comprises the crosslinking moiety.

As another example, an oligonucleotide can be ligated to the template to create a 3′ end that is complementary to the primer. In one aspect, the oligonucleotide comprises the crosslinking moiety. In another aspect, the primer comprises the crosslinking moiety.

The crosslinking moiety can be any chemical moiety which is capable of forming a covalent crosslink between the nucleic acid primer and the target nucleic acid template. For instance, the precursor to the crosslinking moiety can optionally be a coumarin, furocoumarin, or benzodipyrone. Crosslinker moieties useful in the present invention are known to those skilled in the art. For instance, U.S. Pat. Nos. 4,599,303 and 4,826,967 disclose crosslinking compounds based on furocoumarin suitable for use in the present invention. Also, in U.S. Pat. No. 5,082,934, Saba et al describe a photoactivatible nucleoside analogue comprising a coumarin moiety linked through its phenyl ring to a ribose or deoxyribose sugar moiety without an intervening base moiety. In addition, U.S. Pat. No. 6,005,093 discloses non-nucleosidic, stable, photoactive compounds that can be used as photo-crosslinking reagents in nucleic acid hybridization assays. These references are incorporated herein by reference in their entirety for the teaching of crosslinking moieties.

The precursor of the crosslinking moiety can be a coumarin, 7-hydroxycoumarin, 6,7-dihydroxycoumarin, 6-alkoxy-7-hydroxycoumarin, psoralen, 8-methoxypsoralen, 5-methoxypsoralen, 4,5′,8-trimethylpsoralen, 4′-hydroxymethyl-4,5′,8-trimethylpsoralen, and 4′-aminomethyl-4,5′,8-trimethylpsoralen, a haloalkyl coumarin, a haloalkyl furocoumarin, a haloalkyl benzodipyrone, or a derivative thereof. The moiety can be incorporated into a nucleic acid sequence by methods taught in the above referred patents. Compounds containing fused coumarin-cinnoline ring systems are also appropriate for use in the present invention. The crosslinking moiety can be part of a mono-adducted furocoumarin:nucleoside adduct.

The nature of the formation of the covalent bond comprising the crosslink will depend upon the crosslinking moiety chosen. For example, the activation of the covalent bond can occur photochemically, chemically or spontaneously.

A variety of chemistries can be used for covalent crosslinking of DNA strands, including alkylating agents like nitrogen mustard derivatives (Jones et al, 1998) or ultraviolet light-activated agents like derivatives of psoralen (Takasugi et al, 1991). Both classes can be incorporated into synthetic oligonucleotides which are typically used as an anticancer drugs. A sufficient literature exists on photo-activated crosslinkers suitable for a DNA or protein modification. Crosslinkers for this purpose were specially designed to be activated by near UV light (300-400 nm) to prevent damage of biological molecules (in particular DNA) which absorb below this wavelength region.

Light-activated crosslinkers are preferable to alkylators for the purpose of the current method because a crosslinking event can be stimulated at an optimal moment. During the hybridization process DNA strands associate/dissociate in stochastic manner until an equilibrium is reached and most DNA duplexes acquire the desirable configuration. For sufficiently long template DNA this process can take many hours. Light activation of a crosslinker can be done at experimenter's will after a DNA hybridization equilibrium is reached. Alkylators, in contrast to photoactivated crosslinkers, are spontaneous action reagents and may crosslink undesirable temporarily-formed secondary structures of DNA. The crosslinker is preferably attached to the primer rather than dissolved in the system. In the latter case crosslinking may happen randomly at any duplex, whereas in the first case crosslinking will happen only where it is needed.

The primer can carry two modifications—one to connect to the solid support (immobilization agent) and second to connect to a complementary strand (crosslinking agent). Two different strategies can be proposed for an organic synthesis of such a primer. First, a crosslink agent can be introduced into a phosphoramidite derivative of a nucleotide which then is used in a standard phosphoramidite synthesis. The immobilization agent can be a standard C6-amino modification or any other type which are well developed in modern oligonucleotide phosphoramidite synthesis and are known to those skilled in the art. The precaution must be undertaken that the crosslinking moiety must not react with the immolilizing moiety. Second, immobilization and crosslink agents can be combined in one modification and attached to a 5′ end of an oligonucleotide as a last step of a standard synthesis. In both cases it is preferable to create the crosslink at 5′ end of the primer, which leaves more space for DNA polymerase to operate at primer's 3′ end. In designing the primer sequence one has to ensure that a template 3′ end will be long enough to reach the point of crosslink. It is also important to take into account the nucleotide context at the primer's 5′ end at its vicinity, due to the specificity of some crosslink agents. For example, alkylators like nitrogen mustard strongly prefer to crosslink two Gs on the opposite strands in a sequence motif 5′..CG..3′. On the other hand, psoralen, an example of bifunctional photoactivated crosslink agents, prefers to react with pyrimidine residues, mostly with two Ts in a sequence 5′..TA..3′ (Knorre et all, 1989; Knorre et al 1994).

For uniform suppression of mispriming over all primers of a microarray using the current invention it is important to design all primers of the microarray to form correct duplexes with similar melting temperatures (e.g., within ˜5° C.).

2. Primer Extension Conditions

Once the primer-template duplex is stabilized as disclosed herein, the primer extension conditions can be modified to prevent mispriming. For example, stringent hybridization conditions can be used during primer extension. As used herein, “stringent hybridization conditions” refer to conditions that reduce or prevent undesirable secondary structure formation, e.g., hairpin loops, stems, and bulges. Conditions for primer extension can generally be chosen such that the primer remains hybridized to its cognate template sequence (that is, the intended or legitimate template sequence—generally a template sequence fully complementary with the primer) while mismatched and undesirable hybrid structures are fully or partially denatured. The two polynucleotide chains of double-helical DNA can be separated under certain conditions. The transition from double-stranded DNA (dsDNA) to single-stranded DNA (ssDNA) can be referred to as melting, denaturation, or strand separation. The transition from ssDNA to dsDNA is referred to as annealing, renaturation, or, in certain contexts, hybridization. In one aspect, the conditions can be modified to denature non-crosslinked double-stranded DNA. In another aspect, the conditions can be modified to denature non-complementary double-stranded DNA. In another aspect, the conditions can be modified to denature undesirable secondary structures. In another aspect, the conditions can be modified to denature double-stranded DNA with a melting temperature that is lower that the crosslinked primer-template duplex. For example, the conditions can be modified to denature template not crosslinked to primer. Methods for increasing DNA denaturation are known in the art and include, for example, increasing the temperature, reducing salt concentration, and adding denaturing agents.

Denaturation can, but need not, involve complete transition from double strand to single strand, complete melting of strands, or complete strand separation. Thus, partial denaturation or partial strand separation can be referred to as denaturation.

Thus, in one aspect, the method can involve increasing the temperature. The temperature at which the DNA molecules are 50% denatured is referred to as the melting temperature. For increased stringency, the temperature can be maintained, for example, at about 10° C., 9° C., 8° C., 7° C., 6° C., 5° C., 4° C., 3° C., 2° C., 1° C. less than the melting temperature (Tm) of the average primer-template duplex during primer extension. In another aspect the temperature can be maintained at about 40, 45, 50, 55, 60, 65, 70, 75, 80, 85, 90, 95° C. during primer extension.

The method can also comprise decreasing the salt (e.g. chlorides or acetates etc of Na⁺, K⁺, Mg²⁺, M²⁺) concentration. The salt concentration can be, for example, less than about 0.5, 0.4, 0.3, 0.2, 0.1, 0.05, 0.01, 0.001 M.

The method can also involve adding a denaturing agent. Non-limiting examples of denaturing agents include urea and formamide. Both formamide and urea effectively lower the melting point of the DNA duplex structures, allowing the structures to fall apart at lower temperatures. Generally, concentrations of urea or formamide are chosen to give melting temperatures around 50° C. Precaution must be taken when using a denaturing agent that the agent does not cause denaturing of the DNA polymerase enzyme which would be harmful for the SBE process.

3. DNA Sequencing

The disclosed method can be used with any assay where primer extension is used and where denaturation of the primer from the template is not desired. For example, primer extension can be used in the identification of nucleotides in a nucleic acid template (e.g., DNA sequencing). Thus, disclosed are sequencing methods comprising the use of crosslinking to inhibit or reduce mispriming. In one aspect, the disclosed sequencing methods can include one or more steps comprising polymerase-catalyzed incorporation of a nucleotide base into a primer.

A variety of DNA sequencing techniques using primer extension exist and can be used with the disclosed method. Some such techniques differ based on the method of detecting the incorporation of the nucleotide bases into the primer. For example, the pyrophosphate released whenever DNA polymerase adds one of the four deoxynucleoside triphosphates (dNTP's) onto a primer 3′ end can be detected using a chemiluminescent based detection of the pyrophosphate as described in Hyman E. D. (1988, Analytical Biochemistry 174:423-436) and U.S. Pat. No. 4,971,903, which is incorporated herein by reference in its entirety for the teaching of “pyrosequencing.” This approach has been utilized in a sequencing approach referred to as “sequencing by incorporation” as described in Ronaghi (1996, Analytical Biochem. 242:84) and Ronaghi (1998, Science 281:363-365).

A different direct sequencing approach uses dNTPs tagged at the 3′ OH position with four different colored fluorescent tags, one for each of the four nucleotides is described in Metzger, M. L., et al. (1994, Nucleic Acids Research 22:4259-4267). In this approach, the primer/template duplex can be contacted with all four dNTPs simultaneously. Incorporation of a 3′ tagged deoxynucleoside monophosphate (dNMP) can block further chain extension. The excess and unreacted dNTPs can be flushed away and the incorporated DNTP can be identified by the color of the incorporated fluorescent tag. The fluorescent tag can then be removed in order for a subsequent incorporation reaction to occur.

U.S. Pat. No. 6,780,591 describes another sequencing method referred to as “reactive sequencing” or “sequencing-by-synthesis”. This method is based on detection of DNA polymerase catalyzed incorporation of each of the four deoxyribonucleotide types (dGTP, dATP, dTTP, and dCTP) when they are supplied individually and serially to a DNA primer/template system. The DNA primer/template system can comprise a single stranded DNA fragment of unknown sequence, an oligonucleotide primer that forms a matched duplex with a short region of the single stranded DNA, and a DNA polymerase enzyme. The enzyme can either be already present in the template system, or can be supplied together with the dNTP solution. Typically a single dNTP type is added to the DNA primer template system and allowed to react. An extension reaction will occur only when the incoming dNTP base is complementary to the next unpaired base of the DNA template beyond the 3′ end of the primer. While the reaction is occurring, or after a delay of sufficient duration to allow a reaction to occur, the system can be tested to determine whether an additional nucleotide derived from the added dNTP has been incorporated into the DNA primer/template system. A correlation between the dNTP added to the reaction cell and detection of an incorporation signal can identify the nucleotide incorporated into the primer/template. The amplitude of the incorporation signal can identify the number of nucleotides incorporated, and thereby quantify single base repeat lengths where these occur. By repeating this process with each of the four nucleotides individually, the sequence of the template can be directly read in the 5′ to 3′ direction one nucleotide at a time.

4. Detection of Extension

Detection of the polymerase mediated extension reaction and quantification of the extent of reaction can occur by a variety of different techniques, including but not limited to, optical detection of nucleotides tagged with fluorescent or chemiluminescent entities incorporation and microcalorimetic detection of the heat generated by the incorporation of a nucleotide into the extending duplex.

Where the incorporated nucleotide is tagged with a fluorophore, excess unincorporated nucleotide can be removed and the template system illuminated to stimulate fluorescence from the incorporated nucleotide. The fluorescent tag can then be cleaved and removed from the DNA template system before a subsequent incorporation cycle begins. A similar process can be followed for chemiluminescent tags, with the chemiluminescent reaction being stimulated by introducing an appropriate reagent into the system, again after excess unreacted tagged dNTP has been removed; however, chemiluminescent tags are typically destroyed in the process of readout and so a separate cleavage and removal step following detection may not be required. For either type of tag, fluorescent or chemiluminescent, the tag can also be cleaved after incorporation and transported to a separate detection chamber for fluorescent or chemiluminescent detection. In this way, fluorescent quenching by adjacent fluorophore tags incorporated in a single base repeat sequence can be avoided. In addition, this can protect the DNA template system from possible radiation damage in the case of fluorescent detection or from possible chemical damage in the case of chemiluminescent detection. Alternatively the fluorescent tag can be selectively destroyed by a chemical or photochemical reaction. This process eliminates the need to cleave the tag after each readout, or to detach and transport the tag from the reaction chamber to a separate detection chamber for fluorescent detection. The fluorescent tag can also be selectively destroyed by a photochemical reaction with diphenyliodonium ions or related species or by a chemical reaction that specifically destroys the fluorescent tag.

The heat generated by the extension reaction can be measured using a variety of different techniques such as those employing thermopile, thermistor and refractive index measurements. The heat generated by a DNA polymerase mediated extension reaction can be measured. For example, in areaction cell volume of 100 μm³ containing 1 μg of water as the sole thermal mass and 2×10¹¹ DNA template molecules (300 fmol) tethered within the cell, the temperature of the water increases by 1×10⁻³° C. for a polymerase reaction which extends the primer by a single nucleoside monophosphate. This calculation is based on the experimental determination that a one base pair extension in a DNA chain is an exothermic reaction and the enthalpy change associated with this reaction is 3.5 kcal/mole of base. Thus extension of 300 fmol of primer strands by a single base produces 300 fmol×3.5 kcal/mol or 1×10⁻⁹ cal of heat. This is sufficient to raise the temperature of 1 μg of water by 1×10⁻³° C. Such a temperature change can be readily detectable using thermistors (sensitivity ≦10⁻⁴° C.); thermopiles (sensitivity ≦10⁻⁵° C.); and refractive index measurements (sensitivity ≦10⁻⁶° C.).

Thermopiles can used to detect temperature changes. Such thermopiles are known to have a high sensitivity to temperature and can make measurements in the tens of microdegree range in several second time constants. Thermopiles may be fabricated by constructing serial sets of junctions of two dissimilar metals and physically arranging the junctions so that alternating junctions are separated in space. One set of junctions is maintained at a constant reference temperature, while the alternate set of junctions is located in the region whose temperature is to be sensed. A temperature difference between the two sets of junctions produces a potential difference across the junction set which is proportional to the temperature difference, to the thermoelectric coefficient of the junction and to the number of junctions. For optimum response, bimetallic pairs with a large thermoelectric coefficient are desirable, such as bismuth and antimony. Thermopiles may be fabricated using thin film deposition techniques in which evaporated metal vapor is deposited onto insulating substrates through specially fabricated masks. Thermopiles that may be used in the practice of the invention include thermopiles such as those described in U.S. Pat. No. 4,935,345, which is incorporated by reference herein.

Miniature thin film thermopiles produced by metal evaporation techniques, such as those described in U.S. Pat. No. 4,935,345 incorporated herein by reference, can be used to detect the enthalpy changes. Such devices have been made by vacuum evaporation through masks of about 10 mm square. Using methods of photolithography, sputter etching and reverse lift-off techniques, devices as small as 2 mm square may be constructed without the aid of modern microlithographic techniques. These devices contain 150 thermoelectric junctions and employ 12 micron line widths and can measure the exothermic heat of reaction of enzyme-catalyzed reactions in flow streams where the enzyme is preferably immobilized on the surface of the thermopile.

Temperature changes can also be sensed using a refractive index measurement technique. For example, techniques such as those described in Bornhop (1995, Applied Optics 34:3234-323) and U.S. Pat. No. 5,325,170, may be used to detect refractive index changes for liquids in capillaries. In such a technique, a low-power He—Ne laser is aimed off-center at a right angle to a capillary and undergoes multiple internal reflection. Part of the beam travels through the liquid while the remainder reflects only off the external capillary wall. The two beams undergo different phase shifts depending on the refractive index difference between the liquid and capillary. The result is an interference pattern, with the fringe position extremely sensitive to temperature—induced refractive index changes.

The thermal response of the system can be increased by the presence of inorganic pyrophosphatase enzyme which is contacted with the template system along with the dNTP solution. Additional heat is released as the pyrophosphate released from the dNTPs upon incorporation into the template system is hydrolyzed by inorganic pyrophosphatase enzyme. The pyrophosphate released upon incorporation of dNTP's can be removed from the template system and hydrolyzed, and the resultant heat detected, using thermopile, thermistor or refractive index methods, in a separate reaction cell downstream. In this reaction cell, inorganic pyrophosphatase enzyme may be mixed in solution with the dNTP removed from the DNA template system, or alternatively the inorganic pyrophosphatase enzyme may be covalently tethered to the wall of the reaction cell.

Alternatively, the polymerase-catalyzed incorporation of a nucleotide base can be detected using fluorescence and chemiluminescence detection schemes. The DNA polymerase mediated extension is detected when a fluorescent or chemiluminescent signal is generated upon incorporation of a fluorescently or chemiluminescently labeled deoxynucleotide into the extending DNA primer strand. Such tags are attached to the nucleotide in such a way as to not interfere with the action of the polymerase. For example, the tag may be attached to the nucleotide base by a linker arm sufficiently long to move the bulky fluorophore away from the active site of the enzyme.

For use of such detection schemes, nucleotide bases can be labeled by covalently attaching a compound such that a fluorescent or chemiluminescent signal is generated following incorporation of a dNTP into the extending DNA primer/template. Examples of fluorescent compounds for labeling dNTPs include but are not limited to fluorescein, rhodamine, BODIPY (4,4-difluoro-4-bora-3a,4a-diaza-s-indacene) and cyanine dyes (e.g. Cy3, Cy5). See Handbook of Molecular Probes and Fluorescent Chemicals available from Molecular Probes, Inc. (Eugene, Oreg.). Examples of chemiluminescence based compounds that may be used in the sequencing methods of the invention include but are not limited to luminol and dioxetanones (See, Gunderman and McCapra, “Chemiluminescence in Organic Chemistry”, Springer-Verlag, Berlin Heidleberg, 1987).

Fluorescently or chemiluminescently labeled dNTPs can be added individually to a DNA template system containing template DNA annealed to the primer, DNA polymerase and the appropriate buffer conditions. After the reaction interval, the excess DNTP can be removed and the system can be probed to detect whether a fluorescent or chemiluminescent tagged nucleotide has been incorporated into the DNA template. Detection of the incorporated nucleotide can be accomplished using different methods that will depend on the type of tag utilized. For fluorescently-tagged dNTPs, the DNA template system can be illuminated with optical radiation at a wavelength which is strongly absorbed by the tag entity. Fluorescence from the tag can be detected using for example a photodetector together with an optical filter which excludes any scattered light at the excitation wavelength.

Since labels on previously incorporated nucleotides could interfere with the signal generated by the most recently incorporated nucleotide, it is preferred that the fluorescent tag be removed at the completion of each extension reaction. To facilitate removal of a fluorescent tag, the tag can be attached to the nucleotide via a chemically or photochemically cleavable linker using methods such as those described by Metzger, M. L. et al. (1994, Nucleic Acids Research 22:4259-4267) and Burgess, K. et al., (1997, J. Org. Chem. 62:5165-5168) so that the fluorescent tag may be removed from the DNA template system before a new extension reaction is carried out.

The fluorescent tag can also be attached to the dNTP by a photocleavable or chemically cleavable linker. In this case, the tag can be detached following the extension reaction and removed from the template system into a detection cell where the presence, and the amount, of the tag is determined by optical excitation at a suitable wavelength and detection of fluorescence. In this case, the possibility of fluorescence quenching, due to the presence of multiple fluorescent tags immediately adjacent to one another on a primer strand which has been extended complementary to a single base repeat region in the template, is minimized, and the accuracy with which the repeat number can be determined is optimized. In addition, excitation of fluorescence in a separate chamber minimizes the possibility of photolytic damage to the DNA primer/template system.

The signal from the fluorescent tag can also be destroyed using a chemical reaction which specifically targets the fluorescent moiety and reacts to form a final product which is no longer fluorescent. In this case, the fluorescent tag attached to the nucleotide base is destroyed following extension and detection of the fluorescence signal, without the removal of the tag. For example, fluorophores attached to dNTP bases can be selectively destroyed by reaction with compounds capable of extracting an electron from the excited state of the fluorescent moiety thereby producing a radical ion of the fluorescent moiety which then reacts to form a final product which is no longer fluorescent. The signal from a fluorescent tag can also be destroyed by photochemical reaction with the cation of a diphenyliodonium salt following extension and detection of the fluorescence label. The fluorescent tag attached to the incorporated nucleotide base is destroyed, without removal of the tag, by the addition of a solution of a diphenyliodonium salt to the reaction cell and subsequent blue light exposure. The diphenyliodonium salt solution is removed and the reactive sequencing is continued. This method does not require dNTP's with chemically or photochemically cleavable linkers, since the fluorescent tag need not be removed.

The response generated by a DNA polymerase-mediated extension reaction can also be amplified. In this embodiment, the dNTP is chemically modified by the covalent attachment of a signaling tag through a linker that can be cleaved either chemically or photolytically. Following exposure of the dNTP to the primer/template system and flushing away any unincorporated chemically modified dNTP, any signaling tag that has been incorporated is detached by a chemical or photolytic reaction and flushed out of the reaction chamber to an amplification chamber in which an amplified signal can be produced and detected.

A variety of methods can be used to produce an amplified signal. In one such method the signaling tag has a catalytic function. When the catalytic tag is cleaved and allowed to react with its substrate, many cycles of chemical reaction ensue producing many moles of product per mole of catalytic tag, with a corresponding multiplication of reaction enthalpy. Either the reaction product is detected, through some property such as color or absorbency, or the amplified heat product is detected by a thermal sensor. For example, if an enzyme is covalently attached to the dNTP via a cleavable linker arm of sufficient length that the enzyme does not interfere with the active site of the polymerase enzyme. Following incorporation onto the DNA primer strand, that enzyme is detached and transported to a second reactor volume in which it is allowed to interact with its specific substrate, thus an amplified response is obtained as each enzyme molecule carries out many cycles of reaction. For example, the enzyme catalase (CAT) catalyzes the reaction:

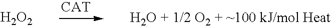

if each dNTP is tagged with a catalase molecule which is detached after dNMP incorporation and allowed to react downstream with hydrogen peroxide, each nucleotide incorporation would generate ˜25 kcal/mol×N of heat where N is the number of hydrogen peroxide molecules decomposed by the catalase. The heat of decomposition of hydrogen peroxide is already ˜6-8 times greater than for nucleotide incorporation, (i.e. 3.5-4 kcal/mol). For decomposition of ˜100-150 hydrogen peroxide molecules the amount of heat generated per base incorporation approaches 1000 times that of the unamplified reaction. Similarly, enzymes which produce colored products, such as those commonly used in enzyme-linked immunosorbent assays (ELISA) can be incorporated as detachable tags. For example the enzyme alkaline phosphatase converts colorless p-nitrophenyl phosphate to a colored product (p-nitrophenol); the enzyme horseradish peroxidase converts colorless o-phenylenediamine hydrochloride to an orange product. Chemistries for linking these enzymes to proteins such as antibodies are well-known to those versed in the art, and can be adapted to link the enzymes to nucleotide bases via linker arms that maintain the enzymes at a distance from the active site of the polymerase enzymes.

An amplified thermal signal can be produced when the signaling tag is an entity which can stimulate an active response in cells which are attached to, or held in the vicinity of, a thermal sensor such as a thermopile or thermistor. Pizziconi and Page (1997, Biosensors and Bioelectronics 12:457-466) reported that harvested and cultured mast cell populations could be activated by calcium ionophore to undergo exocytosis to release histamine, up to 10-30 pg (100-300 fmol) per cell. The multiple cell reactions leading to exocytosis are themselves exothermic. This process is further amplified using the enzymes diamine oxidase to oxidize the histamine to hydrogen peroxide and imidazoleacetaldehyde, and catalase to disproportionate the hydrogen peroxide. Two reactions together liberate over 100 kJ of heat per mole of histamine. For example, a calcium ionophore is covalently attached to the dNTP base via a linker arm which distances the linked calcium ionophore from the active site of the polymerase enzyme and is chemically or photochemically cleavable. Following the DNA polymerase catalyzed incorporation step, and flushing away unincorporated nucleotides any calcium ionophore remaining bound to an incorporated nucleotide can be cleaved and flushed downstream to a detection chamber containing a mast cell-based sensor such as described by Pizziconi and Page (1997, Biosensors and Bioelectronics 12:457-466). The calcium ionophore would bind to receptors on the mast cells stimulating histamine release with the accompanying generation of heat. The heat production could be further amplified by introducing the enzymes diamine oxidase to oxidize the histamine to hydrogen peroxide and imidazoleacetaldehyde, and catalase to disproportionate the hydrogen peroxide. Thus a significantly amplified heat signal would be produced which could readily be detected by a thermopile or thermistor sensor within, or in contact with, the reaction chamber.

The chemiluminescent tag can be attached to the dNTP by a photocleavable or chemically cleavable linker. The tag can be detached following the extension reaction and removed from the template system into a detection cell where the presence, and the amount, of the tag can be determined by an appropriate chemical reaction and sensitive optical detection of the light produced. In this case, the possibility of a non-linear optical response due to the presence of multiple chemiluminescent tags immediately adjacent to one another on a primer strand which has been extended complementary to a single base repeat region in the template, is minimized, and the accuracy with which the repeat number can be determined is optimized. In addition, generation of chemiluminescence in a separate chamber minimizes chemical damage to the DNA primer/template system, and allows detection under harsh chemical conditions which otherwise would chemically damage the DNA primer/template. In this way, chemiluminescent tags can be chosen to optimize chemiluminescence reaction speed, or compatibility of the tagged dNTP with the polymerase enzyme, without regard to the compatibility of the chemiluminescence reaction conditions with the DNA primer/template.

The concentration of the dNTP solution removed from the template system following each extension reaction can be measured by detecting a change in UV absorption due to a change in the concentration of dNTPs, or a change in fluorescence response of fluorescently-tagged dNTPs. The incorporation of nucleotides into the extended template would result in a decreased concentration of nucleotides removed from the template system. Such a change can be detected by measuring the UV absorption of the buffer removed from the template system following each extension cycle.

Extension of the primer strand can be sensed by a device capable of sensing fluorescence from, or resolving an image of, a single DNA molecule. Devices capable of sensing fluorescence from a single molecule include the confocal microscope and the near-field optical microscope. Devices capable of resolving an image of a single molecule include the scanning tunneling microscope (STM) and the atomic force microscope (AFM).

A single DNA template molecule with attached primer can immobilized on a surface and viewed with an optical microscope or an STM or AFM before and after exposure to buffer solution containing a single type of dNTP, together with polymerase enzyme and other necessary electrolytes. When an optical microscope is used, the single molecule can be exposed serially to fluorescently-tagged dNTP solutions and as before incorporation can be sensed by detecting the fluorescent tag after excess unreacted dNTP is removed. Again as before, the incorporated fluorescent tag is preferrably cleaved and discarded before a subsequent tag can be detected. Using the STM or AFM, the change in length of the primer strand is imaged to detect incorporation of the dNTP. Alternatively the dNTP can be tagged with a physically bulky molecule, more readily visible in the STM or AFM, and this bulky tag can be removed and discarded before each fresh incorporation reaction.

### B. MATERIALS

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid is disclosed and discussed and a number of modifications that can be made to a number of molecules including the nucleic acid are discussed, each and every combination and permutation of nucleic acid and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

1. Nucleic Acids

The disclosed nucleic acids, including primers and template, can be made up of for example, nucleotides, nucleotide analogs, or nucleotide substitutes. Non-limiting examples of these and other molecules are discussed herein. As used herein deoxyribonucleotide means and includes, in addition to dGTP, dCTP, dATP, dTTP, chemically modified versions of these deoxyribonucleotides or analogs thereof. Such chemically modified deoxyribonucleotides include but are not limited to those deoxyribonucleotides tagged with a fluorescent or chemiluminescent moiety. Analogs of deoxyribonucleotides that may be used include but are not limited to 7-deazapurine. The present invention additionally provides a method for improving the purity of deoxynucleotides used in the polymerase reaction.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

i. Template

Single-stranded template nucleic acid, such as DNA and RNA, to be sequenced can be obtained from any source and/or can be prepared using any of a variety of different methods known in the art. There are two general types of DNA are particularly useful as templates in the sequencing reactions. Pure single-stranded DNA such as that obtained from recombinant bacteriophage can be used. The use of bacteriophage provides a method for producing large quantities of pure single stranded template. Alternatively, single-stranded DNA can be derived from double-stranded DNA that has been denatured by heat or alkaline conditions, as described in Chen and Subrung, (1985, DNA 4:165); Huttoi and Skaki (1986, Anal. Biochem. 152:232); and Mierendorf and Pfeffer, (1987, Methods Enzymol. 152:556), may be used. Such double stranded DNA includes, for example, DNA samples derived from patients to be used in diagnostic sequencing reactions.

The template DNA can be prepared by various techniques well known to those of skill in the art. For example, template DNA can be prepared as vector inserts using any conventional cloning methods, including those used frequently for sequencing. Such methods can be found in Sambrook et al., Molecular Cloning: A Laboratory Manual, Second Edition (Cold Spring Harbor Laboratories, New York, 1989). Polymerase chain reactions (PCR) can be used to amplify fragments of DNA to be used as template DNA as described in Innis et al., ed. PCR Protocols (Academic Press, New York, 1990).

The amount of DNA template needed for accurate detection of the polymerase reaction will depend on the detection technique used. For example, for optical detection, e.g., fluorescence or chemiluminescence detection, relatively small quantities of DNA in the femtomole range are needed. For thermal detection, quantities approaching one picomole may be required to detect the change in temperature resulting from a DNA polymerase mediated extension reaction.

ii. Primers and Probes

Disclosed are compositions including primers. In certain embodiments the primers can be used to support DNA sequencing reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, DNA duplication, DNA sequencing, PCR reaction, DNA extension, DNA polymerization, RNA transcription, or reverse transcription. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

In enzymatic sequencing reactions, the priming of DNA synthesis is generally achieved by the use of an oligonucleotide primer with a base sequence that is complementary to, and therefore capable of binding to, a specific region on the template DNA sequence. In instances where the template DNA is obtained as single stranded DNA from bacteriophage, or as double stranded DNA derived from plasmids, “universal” primers that are complementary to sequences in the vectors, i.e., the bacteriophage, cosmid and plasmid vectors, and that flank the template DNA, can be used.

Primer oligonucleotides are generally chosen to form highly stable duplexes that bind to the template DNA sequences and remain intact during any washing steps during the extension cycles. Preferably, the length of the primer oligonucleotide is from about 18-60 nucleotides and contains a balanced base composition. The structure of the primer should also be analyzed to confirm that it does not contain regions of dyad symmetry which can fold and self anneal to form secondary structures thereby rendering the primers inefficient. Conditions for selecting appropriate hybridization conditions for binding of the oligonucleotide primers in the template systems will depend on the primer sequence and are well known to those of skill in the art.

2. Polymerase

The disclosed sequencing method can make use of any suitable polymerase to incorporate dNTPs onto the 3′ end of the primer which is hybridized to the template DNA molecule. Such DNA polymerases include but are not limited to Taq polymerase, T7 or T4 polymerase, and Klenow polymerase. For the most rapid reaction kinetics, the amount of polymerase is sufficient to ensure that each DNA molecule carries a non-covalently attached polymerase molecule during reaction.

In addition, reverse transcriptase which catalyzes the synthesis of single stranded DNA from an RNA template can be utilized in the disclosed sequencing methods to sequence messenger RNA (mRNA). Such a method comprises sequentially contacting an RNA template annealed to a primer (RNA primer/template) with dNTPs in the presence of reverse transcriptase enzyme to determine the sequence of the RNA. Because mRNA is produced by RNA polymerase-catalyzed synthesis from a DNA template, and thus contains the sequence information of the DNA template strand, sequencing the mRNA yields the sequence of the DNA gene from which it was transcribed. Eukaryotic mRNAs have poly(A) tails and therefore the primer for reverse transcription can be an oligo(dT). The oligo(dT) primer can be synthesized with a terminal biotin or amino group through which the primer can be captured on a substrate and subsequently hybridize to and capture the template mRNA strand.

DNA polymerases lacking 3′ to 5′ exonuclease activity can be used for SBE to limit exonucleolytic degradation of primers that would occur in the absence of correct dNTPs. In the presence of all four dNTPs, misincorporation frequencies by DNA polymerases possessing exonucleolytic proofreading activity are as low as one error in 106 to 108 nucleotides incorporated as discussed in Echols and Goodman (1991, Annu. Rev. Biochem 60; 477-511); and Goodman et al. (1993, Crit. Rev. Biochem. Molec. Biol. 28:83-126); and Loeb and Kunkel (1982, Annu. Rev. Biochem. 52:429-457). In the absence of proofreading, DNA polymerase error rates are typically on the order of 1 in 10⁴ to 1 in 10⁶. Although exonuclease activity increases the fidelity of a DNA polymerase, the use of DNA polymerases having proofreading activity can pose technical difficulties for the disclosed sequencing methods. Not only will the exonuclease remove any misincorporated nucleotides, but also, in the absence of a correct dNTP complementary to the next template base, the exonuclease will remove correctly-paired nucleotides successively until a point on the template sequence is reached where the base is complementary to the dNTP in the reaction cell. At this point, an idling reaction is established where the polymerase repeatedly incorporates the correct nucleotide and then removes it. Only when a correct dNTP is present will the rate of polymerase activity exceed the exonuclease rate so that an idling reaction is established that maintains the incorporation of that correct nucleotide at the 3′ end of the primer.

A number of T4 DNA polymerase mutants containing specific amino acid substitutions possess reduced exonuclease activity levels up to 10,000-fold less than the wild-type enzyme. For example, Reha-Krantz and Nonay (1993, J. Biol. Chem. 268:27100-17108) report that when Asp 112 was replaced with Ala and Glu 114 was replaced with Ala (D112A/E114A) in T4 polymerase, these two amino acid substitutions reduced the exonuclease activity on double stranded DNA by a factor of about 300 relative to the wild type enzyme. Such mutants can be advantageously used herein for incorporation of nucleotides into the DNA primer/template system.

DNA polymerases which are more accurate than wild type polymerases at incorporating the correct nucleotide into a DNA primer/template can be used. For example, in a (D112A/E114A) mutant T4 polymerase with a third mutation where Ile 417 is replaced by Val (1417V/D112A/E114A), the 1417V mutation results in an antimutator phenotype for the polymerase (Reha-Krantz and Nonay, 1994, J. Biol. Chem. 269:5635-5643; Stocki et al., 1995, Mol. Biol. 254:15-28). This antimutator phenotype arises because the polymerase tends to move the primer ends from the polymerase site to the exonuclease site more frequently and thus proof read more frequently than the wild type polymerase, and thus increases the accuracy of synthesis.

Polymerase mutants that are capable of more efficiently incorporating fluorescent-labeled nucleotides into the template DNA system molecule can be used. The efficiency of incorporation of fluorescent-labeled nucleotides can be reduced due to the presence of bulky fluorophore labels that may inhibit dNTP interaction at the active site of the polymerase. Polymerase mutants that can be advantageously used for incorporation of fluorescent-labeled dNTPs into DNA include but are not limited to those described in U.S. application Ser. No. 08/632,742 filed Apr. 16, 1996 which is incorporated by reference herein.

3. Buffer

The extension reactions can be carried out in buffer solutions that contain the appropriate concentrations of salts, dNTPs and DNA polymerase required for the DNA polymerase mediated extension to proceed. For guidance regarding such conditions see, for example, Sambrook et al., (1989, Molecular Cloning, A Laboratory Manual, Cold Spring Harbor Press, N.Y.); and Ausubel et al. (1989, Current Protocols in Molecular Biology, Green Publishing Associates and Wiley Interscience, N.Y.). As described elsewhere herein, the extension conditions can be adjusted to increase stringency. This can be accomplished, for example, by formulation of buffer used, by addition of components to a reaction (that may already include a buffer), or a combination.

4. Solid Substrate

Either the primer or the template (or both) can be tethered to a solid phase support or substrate to permit the sequential addition of sequencing reaction reagents without complicated and time consuming purification steps following each extension reaction. The primer, template, or combination can be tethered directly or indirectly, covalently or noncovalently using any suitable technique or chemistry. Additionally, the primer, template, or combination can be ligated to an oligonucleotide that is tethered to the substrate.

Numerous techniques and chemistries are known for adhering, associating or coupling molecules to substrates and these can be used with the disclosed primers and templates. Preferably, the primer or template is covalently attached to a solid substrate, such as the surface of a reaction flow cell, a polymeric microsphere, filter material, or the like, which permits the sequential application of sequencing reaction reagents, i.e., buffers, dNTPs and DNA polymerase, without complicated and time consuming purification steps following each extension reaction.

Methods for immobilizing DNA on a solid substrate are well known to those of skill in the art and will vary depending on the solid substrate chosen. For example, DNA can be modified to facilitate covalent or non-covalent tethering of the DNA to a solid substrate. For example, the ends of the DNA strand can be modified to carry a linker moiety for tethering the DNA to a solid substrate. Such linker moieties include, for example, biotin. When using biotin, the biotinylated DNA fragments can be bound non-covalently to streptavidin covalently attached to the solid phase support. Alternatively, an amino group (—NH₂) can be chemically incorporated into the DNA strand and used to covalently link the DNA to a solid phase support using standard chemistry, such as reactions with N-hydroxysuccinimide activated agarose surfaces.

Solid substrates for use in solid-state detectors can include any solid material to which oligonucleotides can be coupled. This includes materials such as acrylamide, cellulose, dextran, nitrocellulose, glass, gold, latex, polyamide, polycarbonate, polystyrene, polyethylene vinyl acetate, polypropylene, polymethacrylate, polyethylene, polyethylene oxide, polysilicates, polyearbonates, quartz, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid substrates can have any useful form including thin films or membranes, beads, bottles, dishes, fibers, optical fibers, woven fibers, chips, compact disks, shaped polymers, particles and microparticles. A chip is a rectangular or square small piece of material. Preferred forms for solid-state substrates are thin films, beads, or chips. Substrates can also be coated with a surface suitable for nucleic acid binding. Non-limiting examples of coatings include epoxides and polyelectrolyte multilayers.

Methods for immobilization of oligonucleotides to solid substrates are well established. Oligonucleotides, including primers, can be coupled to substrates using established coupling methods. Suitable attachment methods are described by Pease et al., Proc. Natl. Acad. Sci. USA 91(11):5022-5026 (1994), Khrapko et al., Mol Biol (Mosk) (JSSR) 25:718-730 (1991), U.S. Pat. No. 5,871,928 to Fodor et al., U.S. Pat. No. 5,654,413 to Brenner, U.S. Pat. No. 5,429,807, and U.S. Pat. No. 5,599,695 to Pease et al, which are incorporated herein by reference in their entirety for these teachings. A method for immobilization of 3′-amine oligonucleotides on casein-coated slides is described by Stimpson et al., Proc. Natl. Acad. Sci. USA 92:6379-6383 (1995). A method of attaching oligonucleotides to solid-state substrates is described by Guo et al., Nucleic Acids Res. 22:5456-5465 (1994). A method of attaching thiol-oligonucleotides to fused silica surface is described in L. A. Chrisey et al (1996). Attaching of amino-oligonucleotides to glass surface is described in J. Li et al (2001) and in E. H. Hansen, H. S. Mikkelsen (1991). A general review of immobilization strategies for biomolecules can be found in Cass T and Ligler FS (1998).

Examples of nucleic acid chips and arrays, including methods of making and using such chips and arrays, are described in U.S. Pat. No. 6,287,768, U.S. Pat. No. 6,288,220, U.S. Pat. No. 6,287,776, U.S. Pat. No. 6,297,006, and U.S. Pat. No. 6,291,193, which are incorporated herein by reference in their entirety for these teachings. Examples of attachment agents are cyanogen bromide, succinimide, aldehydes, tosyl chloride, avidin-biotin, photocrosslinkable agents, epoxides and maleimides. Other standard immobilization chemistries are known by those of skill in the art. A specific example of the use of immobilization strategies for SBE can be found in Aksyonov et al. 2006.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### D. EMBODIMENTS

Provided herein is a method of identifying nucleotides in a nucleic acid template comprising contacting a primer with a nucleic acid template, covalently crosslinking the primer to the template, extending the primer under conditions that denature undesirable secondary structures, and detecting extension of the primer, thereby identifying one or more nucleotides in the template. Generally, the primer can be extended by a single nucleotide type at a time. Thus, the extension of the primer can be detected after each extension attempt by detecting the incorporation of the single nucleotide type. The extension and detection of extension can then be repeated one or more times, thereby identifying a plurality of nucleotides in the template. The primer can be extended under conditions that denature template not crosslinked to primer.

The primer can comprise a crosslinking agent. The crosslinking agent can be photoactivated. For example, the crosslinking agent can be psoralen or a derivative of psoralen, e.g. 8-methoxypsoralen. The primer-template duplex can be exposed to a quantity of UV light sufficient to activate the crosslinking agent. The crosslinking moiety can be any chemical moiety which is capable of forming a covalent crosslink between the nucleic acid primer and the target nucleic acid template. For instance, the precursor to the crosslinking moiety can optionally be a coumarin, furocoumarin, or benzodipyrone. Crosslinker moieties useful in the present invention are known to those skilled in the art. For instance, U.S. Pat. Nos. 4,599,303 and 4,826,967 disclose crosslinking compounds based on furocoumarin suitable for use in the present invention. Also, in U.S. Pat. No. 5,082,934, Saba et al describe a photoactivatible nucleoside analogue comprising a coumarin moiety linked through its phenyl ring to a ribose or deoxyribose sugar moiety without an intervening base moiety. In addition, U.S. Pat. No. 6,005,093 discloses non-nucleosidic, stable, photoactive compounds that can be used as photo-crosslinking reagents in nucleic acid hybridization assays. These references are incorporated herein by reference in their entirety for the teaching of crosslinking moieties.

Primer extension conditions can comprise conditions more stringent than condition under which the template contacts the primer. Stringency can be increased, for example, by adding a denaturing agent. Stringency can be increased, for example, by raising the temperature, lowering the salt concentration, adding a denaturing agent, or any combination thereof. Stringency can be increased, for example, by raising the temperature to about 40, 45, 50, 55, 60, 65, 70, 75, 80, 85, 90, 95° C. Stringency can also be increased by lowering the salt (e.g. chlorides or acetates etc of Na⁺, K⁺, Mg²⁺, Mn²⁺) concentration to less than about 0.5, 0.4, 0.3, 0.2, 0.1, 0.05, 0.01, 0.001 M. 10.

In one aspect, the primer can be immobilized on a solid substrate. In another aspect, the template can be immobilized on a solid substrate. The solid substrates can comprise acrylamide, cellulose, dextran, nitrocellulose, glass, gold, latex, polyamide, polycarbonate, polystyrene, polyethylene vinyl acetate, polypropylene, polymethacrylate, polyethylene, polyethylene oxide, polysilicates, polycarbonates, quartz, teflon, fluorocarbons, nylon, silicon rubber, polyanhydrides, polyglycolic acid, polylactic acid, polyorthoesters, functionalized silane, polypropylfumerate, collagen, glycosaminoglycans, or polyamino acids. The solid substrates can have any useful form including thin films or membranes, slides, beads, bottles, dishes, fibers, optical fibers, woven fibers, chips, compact disks, shaped polymers, particles and microparticles. The substrates can be coated with a surface suitable for nucleic acid binding. Non-limiting examples of coatings include epoxides and polyelectrolyte multilayers.

The nucleic acids, including the primer and the template, can be immobilized onto the solid substrate either covalently or non-covalently.

Extending the primer can comprise contacting the primer-template duplex with a polymerase and a single type of nucleotide under conditions that allow extension of the primer. The nucleotide can comprise a fluorescent moiety, wherein primer extension can be detected by detecting a fluorescent signal emitted by the fluorescent moiety. Primer extension can also be detected by measuring the heat generated by nucleotide incorporation.

Primer extension can also be detected by measuring the concentration of pyrophosphate release by addition of a nucleotide to the primer. In one aspect, the concentration of pyrophosphate can be detected by hydrolyzing the pyrophosphate and measuring heat generated by hydrolysis of the pyrophosphate. Primer extension can also be detected by measuring the refractive index of the buffer. In another aspect, the pyrophosphate is quantitatively converted to ATP by ATP sulfurylase in the presence of adenosine 5′ phosphosulfate. This ATP can drive the luciferase-mediated conversion of luciferin to oxyluciferin that generates visible light in amounts that are proportional to the amount of ATP. The light produced in the luciferase-catalyzed reaction can be detected by a charge coupled device (CCD) camera and seen as a peak in a Pyrogram™. In this case, the light signal is proportional to the number of nucleotides incorporated. Thus, the concentration of pyrophosphate can be detected by measuring the light signal. The polymerase of the provided method can have reduced exonuclease activity.

Also provided herein is a method for stabilizing a nucleic acid duplex for sequencing. The method comprises immobilizing a nucleic acid primer onto a solid substrate, crosslinking the nucleic acid primer to a nucleic acid template, and exposing the primer-template duplex to a deoxyribonucleotide and a polymerase under conditions for the deoxyribonucleotide to be incorporated into the nucleic acid primer if it is complementary to a corresponding base in the nucleic acid template.

Also provided herein is a method of sequencing a nucleic acid template comprising the steps of:
(a) immobilizing a primer onto a solid substrate;
(b) contacting the primer with a nucleic acid template;
(c) crosslinking the desired primer-template duplexes
(d) contacting the primer-template duplex with a DNA polymerase and a single type of deoxyribonucleotide under conditions of increased hybridization stringency which denature uncrosslinked undesired duplexes
(e) removing unincorporated deoxyribonucleotide
(f) detecting extension of the primer and measuring the amount of incorporated nucleotides; and
(g) repeating steps (d) through (f) to determine the nucleotide sequence of the nucleic acid molecule.

### E. KITS

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for DNA sequencing, the kit comprising a primer comprising a crosslnking moiety covalently attached to a solid substrate and suitable buffers comprising one of each type of deoxynucleotide. The kit can also comprise a DNA polymerase.

### F. USES

The disclosed methods and compositions are applicable to numerous areas including, but not limited to, research, diagnosis, and forensic detection relating to DNA sequencing. Other uses are disclosed, apparent from the disclosure, and/or will be understood by those in the art.

### G. DEFINITIONS

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a nucleotide” includes a plurality of such nucleotides, reference to “the nucleotide” is a reference to one or more nucleotides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### H. EXAMPLES

### 1. Example 1

### Sequencing-by-Synthesis of DNA Using on-Surface-Immobilized Primers

DNA polymerase and fluorescently (as well as natural) dNTPs, suffers from false signals, mostly false positive. The origin of false positive signals is due in part to the formation of undesirable structures of DNA that can be extended by polymerase (so called mispriming). The disclosed method involves raising the hybridization stringency (e.g. raising temperature, or lowering salt concentration, or both) for the primer extension reaction to destroy all undesirable structures but to retain the desirable one. To achieve this the desirable DNA structure, the duplex, must be strongly, covalently stabilized.

For duplex stabilization the psoralen modification of the primer is disclosed. Psoralen is known to intercalate into DNA and covalently binds opposite DNA strands of the duplex after UV (˜360 nm) irradiation. Psoralen can be introduced into the primer at the synthesis step. Psoralen-containing precursors for such synthesis are commercially available. The known position of psoralen in the primer ensures that covalent crosslink appears only at the desired position. Additionally, as stabilization of DNA duplex by covalent crosslinking prevents loss of the template, such stabilization is useful for any method manipulating with the same DNA molecule recurrently (like single molecule sequencing-by-synthesis).

### Materials

The template: 5′-GCTCTTCGCGTTGAAGAAGTACAAAATGTCATTAATGCTATGCAGAAAATCTT AGAGTGT-(FAM)-3′ (SEQ ID NO:1). This 60-mer template bears the fluorescein (FAM) label at 3′ end. This allows detection of the template on surface by fluorescent imaging. The template cannot attach to glass surface other than by non-specific adsorption (suppressed) or through hybridization to a complementary primer (promoted). The template was synthesized by Midland Certified Reagent Company, Inc.

The non-psoralen-primer: 5′-(amine C6)-TTCTGCATAGCATTAATGACATTTTGTACTTCTTCAACGC-3′ (SEQ ID NO:2). This 40-mer primer bears one modification at 5′ end —C6-amine. This primer is invisible for fluorescent imaging. Amino link reacts with aldehyde-functionalized glass surface to covalently immobilize the primer. This primer aligns along the template starting from position 8 of the template. The primer was synthesized by Integrated DNA Technologies, Inc.

The psoralen-primer: 5′-[(Psoralen C2), (amine C6)]-XTAATGACATTTTGTACTTCTTCAAC-3′ (SEQ ID NO:3).

This 26-mer primer bears two modifications at 5′ end —C5-amine and C2-psoralen. This primer is invisible for fluorescent imaging. Amino link reacts with aldehyde-functionalized glass surface to covalently immobilize the primer. This primer aligns along the template starting from position 10 of the template. The primer was synthesized by Midland Certified Reagent Company (Midland, Tex.) using an assymetric doubler from Glen Research (Sterling, Va.). The purpose of assymetric doubler is to make a synthetic oligonucleotide, normally an essentially linear polymer molecule, to branch. In this case at position X oligonucleotide branches to carry two modifications at once—C6-amino and C2-psoralen. A list of other such reagents is supplied by Glen Research.

### Methods

Immobilization method. Glass slides were prepared by cleaning in Piranha solution (sulfuric acid and hydrogen peroxide) and then in ammonia hydroxide. Then glass was amino-functionalized by 3-amino-propyl-triethoxysilane. Then glass was aldehyde functionalized by glutaraldehyde. Then primers were immobilized in a shape of round spots ˜1 mm diameter. Each spot carries only one primer. Surface density of a primer is ˜10 fmole/mm².

Measurement method: Epifluorescent imaging. Excitation light was 488 nm laser radiation from cw Ar laser ˜100 mW. Glass slide was observed by CCD camera through band pass filter 500 to 550 nm.

UV-irradiation: Xe lamp light filtered by UG-3 band pass filter (˜300 to 400 nm). Light flux at a slide was ˜3 W/mm². Duration ˜5 min.

Fluorescence erasure: by treating a slide with solution of diphenyliodonium chloride accompanied with 488 nm irradiation.

Primer extension: T4 bacteriophage DNA polymerase was a specially designed mutant form with amino acid substitutions D112A/E114A/L412M (Linda Reha-Krantz). Fluorescein-labeled analogs of dNTPs were from Perkin Elmer.

### Results

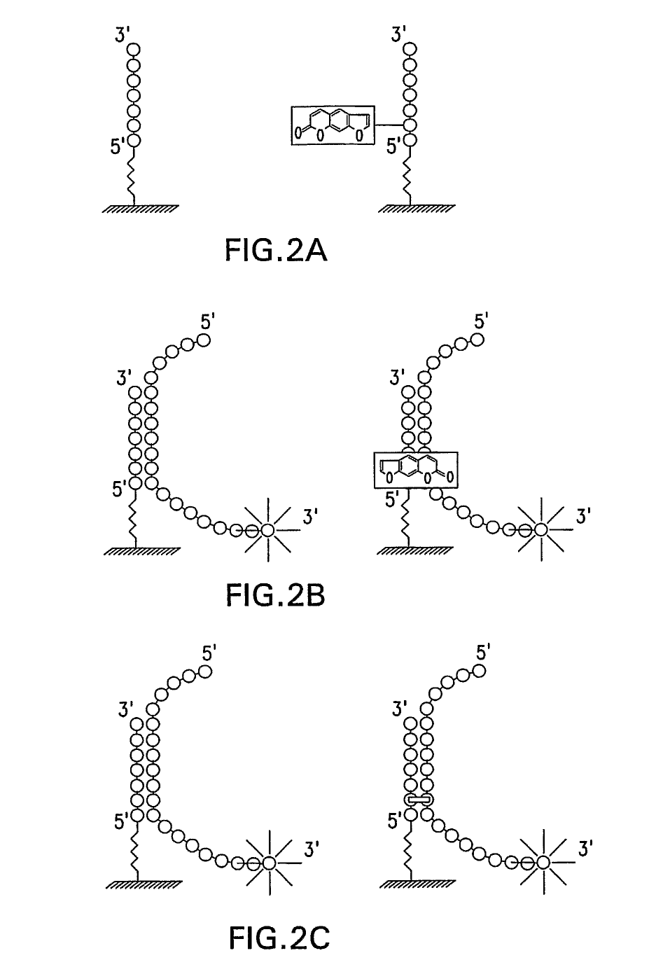

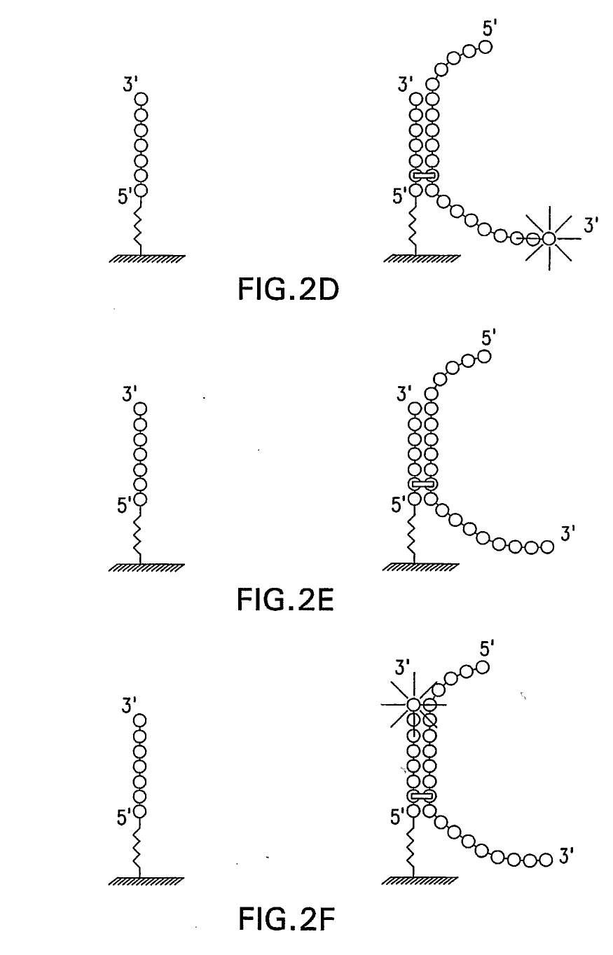

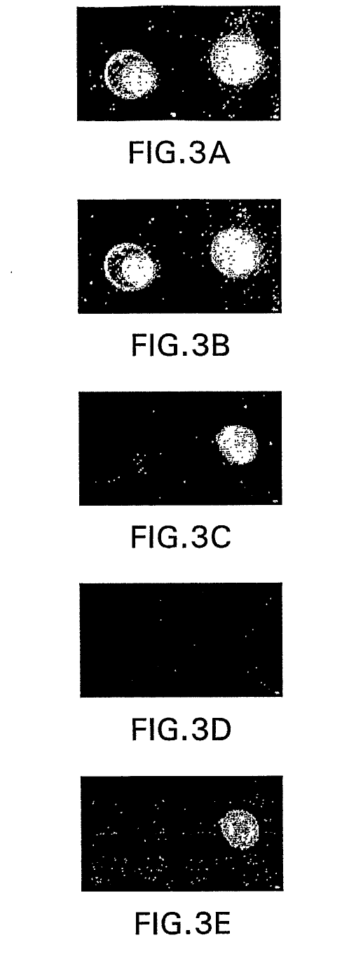

As diagramed in FIG. 2, primers were immobilized onto glass surface, wherein they were not yet visible by fluorescence (FIG. 2A). Once the fluorescent template was captured from solution by the primers, they can be detected by fluorescence (FIG. 2B). Indeed, glowing spots were visible after treating the glass surface with solution of the template (FIG. 3B). UV irradiation is expected to activate psoralen which crosslinked two strands of DNA duplex (FIG. 2C). UV irradiation had no visual effect at this point (FIG. 3C). 95° C. treatment removes all template from non-psoralen primer (column 1, FIG. 2D), but psoralen-primer retains crosslinked template (column 2, FIG. 2D). After 95° C. treatment, non-psoralen primer spot disappeared, but psoralen-primer spot was still visible (correspondingly columns 1 and 2, FIG. 3D). Fluorescence was abolished by DPI treatment (FIG. 2E, 3E). The addition of DNA polymerase and match Fluor-dNTP can result in the incorporated fluorescein-dNTP into DNA substrate (FIG. 2F). In fact, the psoralen-primer spot appeared again after treatment with DNA polymerase and corresponding fluorescein-dNTP (FIG. 3F).

### I. REFERENCES

Aksyonov, S. A. Bittner, M. Bloom, L. B. Reha-Krantz, L. J. Gould, I. R. Hayes, M. A. Kiernan, U. A. Niederkofler, E. E. Pizziconi, V. Rivera, R. S. Williams, D. J. B. Williams, P. “Multiplexed DNA sequencing by synthesis”, Analytical Biochemistry 348 (2006) 127-138
Cass T, Ligler F S, eds. Immobilized biomolecules in Analysis: Oxford University Press, 1998.
Chrisey, L. A. Lee, G. U. O'Ferrall, C. E. Covalent attachment of synthetic DNA to self-assembled monolayer films, Nucleic Acids Res. 24 (1996) 3031-3039.
Denny W A, ed. New developments in the use of nitrogen mustard alkylating agents as anticancer drugs, //In “Advances in DNA Sequence-Specific Agents” series, Eds. Graham B. Jones and Manlio Palumbo, v.3, JAI Press 1998, p. 157.
Gemignani, F. Landi, S. Canzian, F. A review of strategies based on primer extension (PEX) technology, //Minerva Biotechnol. 14 (2002) 231-236.
Hansen, E. H. Mikkelsen, H. S. Enzyme-immobilization by the glutardialdehyde procedure. An investigation of the effects of reducing the Schiff-bases generated, as based on studying the immobilization of glucose oxidase to silanized controlled pore glass, Analytical Letters 24 (1991) 1419-1430.
Knorre D G, Vlassov V V. Affinity Modification of Biopolymer: CRC Press, Inc., 1989.
Knorre D G, Vlassov V V, Zarytova V F, Lebedev A V, Fedorova O S. Design and Targeted Reactions of Oligonucleotides Derivatives: CRC Press, 1994.
Li, J. Wang, H. Zhao, Y. Cheng, L. He, N. Lu, Z. Assembly method fabricating linkers for covalently bonding DNA on glass surface, Sensors 1 (2001) 53-59.
Mitra, R. D. Shendure, J. Olejnik, J. Krzymanska-Olejnik, E. Church, G. M. Fluorescent in situ sequencing on polymerase colonies //Anal. Biochem. 320 (2003) 55-65.
Nikiforov, T. T. Rendle, R. B. Goelet, P. Rogers, Y.-H. Kotewicz, M. L. Anderson, S. Trainor, G. L. Knapp, M. R. Genetic bit analysis: a solid phase method for typing single nucleotide polymorphisms //Nucleic Acids Res. 22 (1994) 4167-4175.
Sergei A. Aksyonov, Linda J. Reha-Krantz, Raul Rivera and Peter Williams. Suppressing the False Signals in Primer Extension Reaction by Enzymatic Scavenging of Contaminating Deoxyribonucleoside Triphosphates //in preparation.
Sergei A. Aksyonov, Linda B. Bloom, Linda Reha-Krantz, Ian R. Gould, Mark A. Hayes, Urban A. Kiernan, Eric E. Niederkofler, Raul S. Rivera, Daniel J. B. Williams, Peter Williams Fluorescent Sequencing-by-Extension of DNA on Microarrays //Presentation on TIGR-XVI International conference, Washington, D.C., Sep. 27-30, 2004.
Takasugi M, Guendouz A, Chassignol M, Lhomme J L D, Thuong N T, Helene C. Sequence-Specific Photo-Induced Crosslinking of the Two Strands of Double-Helical DNA by a Psoralen Covalently Linked to a Triple Helix-Forming Oligonucleotide. //Proc. Natl. Acad. USA 1991; 88:5602-5606.

J. Sequences

SEQ ID NO:1

GCTCTTCGCGTTGAAGAAGTACAAAATGTCATTAATGCTATGCAGAAAAT
CTTAGAGTGT

SEQ ID NO:2

TTCTGCATAGCATTAATGACATTTTGTACTTCTTCAACGC

SEQ ID NO:3

XTAATGACATTTTGTACTTCTTCAAC

What is claimed is:

1. A method of identifying nucleotides in a nucleic acid template comprising contacting a primer with a nucleic acid template, crosslinking the primer to the template, and extending the primer under conditions that denature undesirable secondary structures and detecting extension of the primer, thereby identifying one or more nucleotides in the template.

2. The method of claim 1, wherein the primer is extended by a single nucleotide type at a time, wherein extension of the primer is detected after each extension by a single nucleotide type, and wherein extension and detection of extension are repeated one or more times, thereby identifying a plurality of nucleotides in the template.

3. The method of claim 1, wherein the primer comprises a crosslinking agent.

4. The method of claim 3, wherein the crosslinking agent is photoactivated.

5. The method of claim 4, wherein the crosslinking agent is psoralen.

6. The method of claim 4, wherein the primer-template duplex is exposed to a quantity of UV light sufficient to activate the crosslinking agent.

7. The method of claim 1, wherein primer extension conditions comprise conditions more stringent than condition under which the template contacts the primer.

8. The method of claim 7, wherein stringency is increased by raising the temperature.

9. The method of claim 7, wherein stringency is increased by lowering the salt concentration.

10. The method of claim 7, wherein stringency is increased by adding a denaturing agent.

11. The method of claim 10, wherein the denaturing agent is urea or formamide.

12. The method of claim 1, wherein the primer is immobilized on a solid substrate.

13. The method of claim 1, wherein the template is immobilized on a solid substrate.

14. The method of claim 12, wherein the solid substrate is glass or silica.

15. The method of claim 1, wherein extending the primer comprises contacting the primer-template duplex with a polymerase and a single type of nucleotide under conditions that allow extension of the primer.

16. The method of claim 15, wherein the nucleotide comprises a fluorescent moiety, and wherein primer extension is detected by detecting a fluorescent signal emitted by the fluorescent moiety.

17. The method of claim 1, wherein primer extension is detected by measuring the heat generated by nucleotide incorporation.

18. The method of claim 1, wherein primer extension is detected by measuring the concentration of pyrophosphate release by addition of a nucleotide to the primer,

19. The method of claim 18, wherein the concentration of pyrophosphate is detected by hydrolyzing the pyrophosphate and measuring heat generated by hydrolysis of the pyrophosphate.

20. The method of claim 18, wherein the pyrophosphate is converted into ATP by ATP sulfurylase in the presence of adenosine 5′ phosphosulfate, wherein ATP drives the luciferase-mediated conversion of luciferin to oxyluciferin that generates visible light in amounts that are proportional to the amount of ATP, wherein the concentration of pyrophosphate is detected by measuring visible light.

21. The method of claim 1, wherein primer extension is detected by measuring the refractive index of the buffer.

22. The method of claim 1, wherein the polymerase has reduced exonuclease activity.

23. The method of claim 13, wherein the solid substrate is glass or silica.


SEQUENCE LISTING (3 sequences)

SEQ ID NO: 1 - DNA, 60 bp, Artificial Sequence
  Description of Artificial Sequences; note = synthetic construct
gctcttcgcgttgaagaagtacaaaatgtcattaatgctatgcagaaaatcttagagtgt

SEQ ID NO: 2 - DNA, 40 bp, Artificial Sequence
  Description of Artificial Sequences; note = synthetic construct
ttctgcatagcattaatgacattttgtacttcttcaacgc

SEQ ID NO: 3 - DNA, 25 bp, Artificial Sequence
  Description of Artificial Sequences; note = synthetic construct
taatgacattttgtacttcttcaac